(19) United States
(12) Patent Application Publication
(10) Pub. No.: US 2004/0010081 A1
(43) Pub. Date: Jan. 15, 2004

(54) Novel strategy for synthesizing polymers in surfaces

(76) Inventors: Ramon Guimil, Heidelberg (DE); Peer F. Stahler, Mannheim (DE); Matthias Scheffler, Hirschberg/Leutershausen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/362,568

(22) PCT Filed: Aug. 24, 2001
(86) PCT No.: PCT/EP01/09813

(30) Foreign Application Priority Data
Aug. 24, 2000 (DE) 10041542.3

(51) Int. Cl. C07H 21/04; C07K 1/02; C07H 5/04; C08B 37/00

(52) U.S. Cl. 525/54.2; 530/333; 536/55.3; 536/25.4

(57) ABSTRACT

The invention relates to a novel method for synthesising polymers, particularly biopolymers such as nuclear acids, peptides and saccharides, on the surface of solid supports. The inventive method enables improved quality polymers to be produced at a lower cost. Novel structures of support-bound polymers are also disclosed.

The invention relates to a novel process for synthesizing polymers, especially biopolymers such as nucleic acids, peptides and saccharides, on the surface of solid supports. The process of the invention makes it possible to prepare polymers of improved quality, and to reduce costs. In addition, novel structures of polymers bound to supports are provided.

The synthesis of biopolymers such as, for example, DNA or peptides on a solid support usually takes place by directed condensation of bifunctional monomer units.

The starting point of the synthesis is the elimination of a protective group on a functional group bound to a support. The first bifunctional monomer in activated form is then coupled to the functional group. Elimination of the monomer's protective group is followed by renewed coupling of an activated synthon to the functional group which has been liberated.

Peptides and peptide-nucleic acids (PNA) are synthesized by assembling either from the N to the C terminus or from the C to the N terminus, while the synthesis of nucleic acids such as RNA, LNA or DNA takes place from the 3′ end to the 5′ end or in the reverse direction. The polymers are accordingly assembled perpendicular to the surface, as depicted diagrammatically in FIG. 1. The overall yield corresponds to the product of the yield in the individual coupling steps. The coupling yield in each individual condensation step is crucial for the quality of the polymer. This quality in turn influences the suitability thereof for the subsequent applications. A crucial part is played in this connection not only by optimization of the coupling parameters, of the quality of the deprotection procedure and of the activators but also by the nature of possible spacers which are used and their physicochemical properties.

The object on which the invention was based was to develop novel strategies for synthesizing polymers on solid supports which are advantageous compared with the aforementioned method. This object is achieved by a process comprising the steps:

(a) provision of a solid support with a reactive group,

(b) coupling of a trifunctional synthon to the reactive group of the support, where the trifunctional synthon is a compound having the general formula (I):

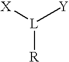

where X and Y are mutually orthogonal protective groups,

L is a unit for synthesizing a polymer, and

R is a group which can, where appropriate after activation, be coupled to the reactive group of the support,

(c) directed assembly of a polymer part-strand starting from the protective group X and

(d) directed assembly of a polymer part-strand starting from the protective group Y.

The process of the invention comprises a combination of two contrary directions of synthesis and shifts the starting point of the polymer synthesis from one of the ends into the interior, for example the middle of the polymers to be synthesized, without influencing the overall molecular orientation. For this purpose, the starting point used for the synthesis is, instead of a bifunctional synthon, a trifunctional synthon which is provided with orthogonal protective groups. A diagrammatic depiction of the process of the invention is depicted in FIG. 2.

In the embodiment shown in FIG. 2, which is based on oligonucleotide chemistry, the strategy requires the use of 5′-protected synthons M, e.g. phosphoramidites, and the inverse 3′-protected synthons M′, e.g. phosphoramidites, in order to obtain a molecule with a uniform orientation (head-tail or tail-head polymer). As a modification thereof, the process of the invention also, of course, makes possible the specific synthesis of head-head (5′-5′) or tail-tail (3′-3′) polymers.

Step (a) in the process of the invention comprises the provision of a solid support having a reactive group. Suitable solid supports are inorganic or organic materials, e.g. functionalized controlled pore glass (CPG), other glasses such as Foturan, Pyrex or usual sodalime glasses, metallic supports such as, for example, silicon, or organic resins such as, for example, Tentagel. The support is particularly preferably a chip employed for synthesizing polymer arrays.

The trifunctional synthon (I) is preferably coupled to the support via a spacer. The use of a spacer has the advantage that the polymer is further away from the surface of the solid support, so that the influences thereof are diminished, so that the immobilized polymer can undergo quasi-homogeneous reactions. The spacer can be assembled by using amino acid monomers or cyanoethyl- or otherwise protected phosphoramidites or H-phosphonates. Examples of suitable spacer units are known in the state of the art.

Step (b) of the process of the invention comprises the coupling of the trifunctional synthon (I) to the reactive group of the support which is derivatized where appropriate by a spacer. Examples of suitable reactive groups are hydroxyl, thiol, carboxyl, aldehyde, epoxy, amino etc. Coupling of the synthon (I) to the reactive group of the support takes place in a conventional way via the group R, which is a group which can be coupled, where appropriate after activation, to the reactive group of the support. Numerous examples of the group R are known from the state of the art, such as, for example, carboxyl or amidite.

An essential feature of the trifunctional synthon (I) is the presence of two orthogonal protective groups X and Y. These protective groups are selected from protective groups known for the solid-phase synthesis of polymers, for example nucleic acids and peptides, with the proviso that assembly of a polymer based on the use of a first one of the two protective groups is possible without influencing the second protective group, i.e. the two protective groups are mutually orthogonal. X and Y are preferably protective groups which can be eliminated by chemical, photochemical or enzymatic reactions, the elimination liberating a reactive group, e.g. a hydroxyl or amino group. Examples of suitable protective groups are acid-labile protective groups such as, for example, dimethoxytrityl (DMT), MMT, pixyl, Fpmp etc., base-labile protective groups, such as, for example, benzyl, benzoyl, isobutyryl, phenoxyacetyl, levulinyl etc., oxidation- or/and reduction labile protective groups, photolabile protective groups such as, for example, NVOC, NPPOC, protective groups which can be eliminated catalytically, e.g. by Pd, such as, for example, allyl, AOC, protective groups which can be eliminated by fluoride, such as, for example, TMS or derivatives thereof, e.g. TBDMS. It is additionally possible to employ combinations of pluralities of said principles via multistage protective group concepts. Preferred examples of combinations of orthogonal protective groups are a protective group which can be eliminated catalytically, such as AOC, and a trityl group.

The group L of the compound (I) is a unit for synthesizing polymers, preferably a monomeric unit, but it is also possible to employ oligomeric units, e.g. dimeric or trimeric units. L is preferably selected from units for synthesizing nucleic acids such as DNA, RNA or LNA nucleic acid analogs, such as, for example, PNA, peptides and saccharides. In a particularly preferred embodiment, L is a monomeric unit for RNA synthesis. Trifunctional RNA monomers of this type (although without protective groups) also occur in nature during splicing of mRNA molecules and are capable of forming so-called lariat structures. One example of a branching unit of this type is shown in FIG. 3, where B′ is a nucleobase, X is an acid-labile protective group such as DMT, MMT or pixyl, Y is a protective group orthogonal thereto, e.g. a photolabile protective group such as NVOC or NPPOC, and R is a phosphoramidite group.

Steps (c) and (d) of the process of the invention comprise the directed assembly of two polymer part-strands, in each case starting from the protective group X and the protective group Y, with two separate polymer part-strands being assembled through use of the suitable protective group chemistry. In a preferred embodiment of the process of the invention there is initially synthesis of the first part-polymer. There are several possible applications for the following steps: on the one hand, it is possible at the start of the second part-synthesis to return to the compound (I) as starting molecule. On the other hand, it is possible to hybridize a nucleic acid having a suitable sequence onto the first part-sequence with a 3′-5′ orientation, so that the latter functions in cooperation with the trifunctional molecule as 3′ primer and can be enzymatically extended, for example with the aid of polymerases or ligases (primer extension). In the case of an oligomer which is assembled from 31 monomers, the process of the invention then assembles two polymer part-strands 15 monomer units long. This leads to a uniform polymer structure with improved quality.

This improved quality results in the possibility of carrying out smaller batches, and smaller sites for a particular polymer species being sufficient in the synthesis of arrays or chips. This leads to a more favorable signal/noise ratio and to a considerable reduction in costs.

The quality can moreover be further increased because all half-sequences which comprise a free 3′-hydroxyl function on the trifunctional molecule are assembled in a basic medium on a hydrolysis-labile socket, so that they can be eliminated from the solid support during the deprotection procedure. This discrimination of terminal sequences leads to a further improvement in the signal/noise ratio.

The horizontal arrangement of the polymers on the surface of the support moreover increases the mobility of the molecules and diminishes surface influences. This leads in the case of nucleic acids to an improved hybridization, because the additional degrees of freedom favor the spatial preorganization which precede the molecular recognition process in strand pairing. This advantage is also important especially in the production of arrays or biochips. Important areas of application of the process of the invention are therefore molecular diagnosis in the areas of human and veterinary medicine, the development of novel pharmaceutically active substances, fundamental biological, biochemical and bioorganic research, in vitro selection, food analysis, biotechnology, and high-throughput methods for screening active substances in combinatorial chemical studies.

A particularly preferred application is in vitro selection, e.g. of ribozymes, i.e. RNA molecules or RNA analogs capable of the sequence-specific cleavage of other nucleic acid molecules. Such selection experiments can be carried out with a target molecule immobilized on a solid support. In newer approaches this entails immobilization of a smaller, less diverse pool of target molecules in an appropriately spatially resolved addressable array. This method is, however, problematic because at present no arrays with a sufficiently large number of probes are available, and they cannot be adapted flexibly to the particular requirements. The T-shaped structures of the invention afford a considerable advantage in this connection because of their additional degrees of freedom, especially in the selection for catalytic activity.

It is moreover possible to employ as trifunctional molecules not only the synthons mentioned but also other substances, e.g. any linear or branched aliphatic, olefinic or aromatic trifunctional hydrocarbons which are optionally substituted by heteroatoms. It is likewise possible to use ethylene and propylene glycol derivatives.

The invention further relates to a support for the solid-phase synthesis of polymers of the general formula (II)

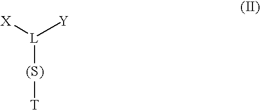

in which T is a solid support as indicated above, L, X and Y are as defined above, and (S) is a spacer which is present where appropriate.

The invention also further relates to a support with an immobilized polymer of the general formula (III)

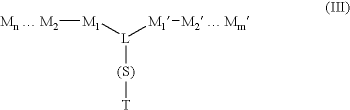

where T is a solid support as indicated above, L and (S) are as indicated above, and M₁ . . . Mₙ and M₁′ . . . Mₘ′ are monomeric units of a polymer, where n+m is preferably 5 to 100, particularly preferably 5 to 50.

The compounds (I) of the invention are prepared by, for example, starting from one of the ribonucleosides (A, G, C, U) by protecting the exocyclic amino functions by means of Jones transient protection with, for example, phenoxyacetyl. The primary 5′-hydroxyl group is then reacted for example with DMT.

Introduction of the functionality orthogonal to the first protective group, e.g. NPPOC, results in a mixture of isomers. After separation of the isomers, the 2′ position is phosphitylated.

1. A process for synthesizing polymers on a solid support comprising the steps:
(a) provision of a solid support with a reactive group,
(b) coupling of a trifunctional synthon to the reactive group of the support, where the trifunctional synthon is a compound having the general formula (I): 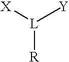
where X and Y are mutually orthogonal protective groups,
L is a unit for synthesizing a polymer, and
r is a group which can, where appropriate after activation, be coupled to the reactive group of the support,
(c) directed assembly of a polymer part-strand starting from the protective group X and
(d) directed assembly of a polymer part-strand starting from the protective group Y.

2. The process as claimed in claim 1, characterized in that the coupling of the trifunctional synthon to the support takes place via a spacer.

3. The process as claimed in claim 1 or 2, characterized in that X and Y are protective groups which can be eliminated by chemical, photochemical or enzymatic reactions.

4. The process as claimed in any of claims 1 to 3, characterized in that the combination of the protective groups X and Y comprises a protective group which can be eliminated catalytically and a trityl protective group.

5. The process as claimed in any of claims 1 to 4, characterized in that L is selected from units for synthesizing nucleic acids, nucleic acid analogs, peptides and saccharides.

6. The process as claimed in any of claims 1 to 5, characterized in that step (d) is carried out as chemical synthesis or as enzymatic synthesis.

7. A support for the solid-phase synthesis of polymers of the general formula (II) 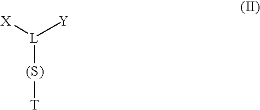
in which T is a solid support, L, X and Y are as defined in claim 1, and (S) is a spacer which is present where appropriate.

8. A support with immobilized polymer of the general formula (III): 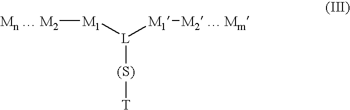
in which T is a solid support, L is as defined in claim 1, (S) is a spacer which is present where appropriate, and M₁ . . . Mₙ and M₁′ . . . Mₘ′ are monomeric units of a polymer.
